(19) United States
(12) Patent Application Publication
(10) Pub. No.: US 2026/0355119 A1
(43) Pub. Date: Nov. 12, 2026

(54) INTRANASAL FORMULATION FOR PULMONARY DELIVERY OF NANOPARTICLES, COMPOSITIONS, USES, AND MANUFACTURING METHODS THEREOF

(71) Applicant: GLOBAL LIFE SCIENCES SOLUTIONS CANADA ULC, Vancouver (CA)

(72) Inventors: Sams Mohammad Anowar Sadat, Vancouver (CA); Anitha Thomas, Vancouver (CA)

(21) Appl. No.: 19/515,083

(22) PCT Filed: Aug. 28, 2024
(86) PCT No.: PCT/US2024/044193

§ 371 Date: Feb. 27, 2026

Related U.S. Application Data
(60) Provisional application No. 63/628,884, filed on Aug. 31, 2023.

(51) Int. Cl. A61K 9/51; A61K 9/00; A61K 9/127; A61K 39/00; A61K 39/215; A61K 47/18; A61K 47/26; A61K 47/38; A61K 47/42; A61K 48/00

(52) U.S. Cl. CPC A61K 9/5123; A61K 9/0043; A61K 9/127; A61K 9/5146; A61K 9/5192; A61K 39/215; A61K 47/18; A61K 47/183; A61K 47/26; A61K 47/38; A61K 47/42; A61K 48/0041; A61K 48/005; A61K 2039/53; A61K 2039/543

(57) ABSTRACT

The invention provides intranasal formulation for pulmonary delivery of nanoparticles, compositions, uses, and manufacturing methods thereof. In one aspect, an intranasal formulation for intranasal or intrapulmonary administration includes a viscosity agent, an epithelial adherence agent, and a foaming agent. The viscosity agent may include carboxymethylcellulose, the epithelial adherence agent may include poly-lysine, and the foaming agent may include gelatin hydrolysate.

### BACKGROUND

Pulmonary gene therapy and mucosal immunization remain challenging as an approach to treat inherited lung diseases, pulmonary hypertension, asthma, and cancers.

Specifically, there are some genetic disorders (e.g., al-antitrypsin-deficiency, cystic fibrosis) resulting from a lack of protein expression or faulty protein, which could be restored by gene therapy.

Recent clinical successes of mRNA-based vaccines indicate that lipid nanoparticles (LNPs) may be used to deliver nucleic acids to humans, however, the systemic therapeutic application of currently available LNP- and non-LNP-based nanoparticulate platforms have been investigated mostly for liver diseases because of their favorable accumulation tendency into the liver, which restricts their access to other organs of the body.

LNPs undergo tremendous shear force when nebulized or aerosolized, which makes pulmonary delivery strategies elusive. In addition, the presence of airway mucus in the lumen impedes LNP contacting the epithelial cell layer of the lungs. Even when the LNP is in contact with the epithelial layer, the epithelial barrier impedes the entry of the payloads into the lung cells. Thus, any LNP solution would need to address that problem to achieve pulmonary gene expression.

Until now, there has not been an efficient and non-toxic formulation composition available to deliver therapeutic LNP into the lung directly as a non-hepatic target.

### BRIEF SUMMARY

In one aspect, the invention provides an intranasal formulation for intranasal or intrapulmonary administration including a viscosity agent, an epithelial adherence agent, and a foaming agent.

In some embodiments, the viscosity agent includes carboxymethylcellulose.

In some embodiments, the epithelial adherence agent includes poly-lysine. In some embodiments, the poly-lysine has a molecular weight of about 15,000 Da to about 30,000 Da.

In some embodiments, the foaming agent includes gelatin hydrolysate.

In some embodiments, the formulation further includes a cryoprotectant. The cryoprotectant may include an emulsifying agent, a sugar, or a combination thereof. In some embodiments, the cryoprotectant includes tris(hydroxymethyl)aminomethane and sucrose.

In some embodiments, the formulation further an amino acid. The amino acid may include arginine, leucine, histidine, glycine, or a combination thereof.

In some embodiments, the formulation includes, based on grams per 100 millimeters (mL) of the formulation (% wt/v), about 0.1 to about 2% wt/v of the viscosity agent, about 0.01 to about 0.6% wt/v of the epithelial adherence agent, and about 1 to about 4 grams % wt/v of the foaming agent,

In some embodiments, the formulation further includes sucrose that is about 10 to about 20 grams per 100 mL of the formulation.

In some embodiments, the formulation further includes an amino acid that is about 0.05 to about 0.4 grams per 100 mL of the formulation.

In some embodiments, the formulation includes an aqueous buffer.

In some embodiments, the formulation is administered intranasally.

In some embodiments, the formulation is administered intratracheally.

In some embodiments, the formulation is in aerosolized or nebulized form.

In some embodiments, the formulation further includes a lipid nanoparticle, liposome, polymeric nanoparticle, or lipomiceller composition. The lipid nanoparticle, liposome, polymeric nanoparticle, or lipomiceller composition may include a nucleic acid, a peptide, or a protein.

In another aspect, the invention provides a composition for intranasal or intrapulmonary administration including the intranasal formulation and a lipid nanoparticle composition comprising: (a) an ionizable lipid; (b) a structural lipid; (c) a cholesterol; and (d) a stabilizing agent.

In some embodiments, the ionizable lipid includes DODMA, DLin-MC3-DMA, DLin-KC2-DMA, butanoic acid, BOCHD-C3-DMA, C12-200, PNI 127, PNI 516, PNI 580, PNI 726, PNI 728, PNI 730, or a combination thereof.

In some embodiments, the structural lipid is neutrally charged, positively charged, or negatively charged. The structural lipid may include diacylphosphatidylcholines, diacylphosphatidylethanolamines, diacylphosphatidylglycerols, ceramides, sphingomyelins, dihydrosphingomyelins, cephalins, cerebrosides, or a combination thereof. In some embodiments, the structural lipid may include distearoylphosphatidylcholine, dioleoylphosphatidylcholine, dipalmitoylphosphatidylcholine, dioleoyl-phosphatidylethanolamine, palmitoyloleoylphosphatidylcholine, 1-stearoyl-2-oleoyl-sn-glycero-3-phosphocholine, palmitoyloleoyl-phosphatidylethanolamine, dioleoyl-phosphatidylethanolamine 4-(N-maleimidomethyl)-cyclohexane-1-carboxylate, dipalmitoyl phosphatidyl ethanolamine, dimyristoylphosphoethanolamine, distearoyl-phosphatidylethanolamine, 1,2-dipalmitoyl-sn-glycero-3-phosphoethanolamine-N-methyl, 1,2-dipalmitoyl-sn-glycero-3-phosphoethanolamine-N,N-dimethyl, 1,2-dielaidoyl-sn-glycero-3-phosphoethanolamine, 1-stearoyl-2-oleoyl-phosphatidyethanol amine, 1,2-dielaidoyl-sn-glycero-3-phophoethanolamine, distearoylphosphatidylcholine, dioleoylphosphatidylglycerol, dipalmitoylphosphatidylglycerol, palmitoyloleyolphosphatidylglycerol, cardiolipin, phosphatidylinositol, diacylphosphatidylserine, diacylphosphatidic acid, monosialoganglioside GM1, or a combination thereof.

In some embodiments, the sterol comprises cholesterol, beta-sitosterol, 20-alpha-hydroxysterol, phytosterol, or a combination thereof.

In some embodiments, the lipid nanoparticle composition comprises about 25 to about 50 mol % ionizable lipid, about 10 to about 50 mol % structural lipid, about 20 to about 50 mol % sterol, and about 0 to about 3 mol % stabilizing agent.

In yet another aspect, the invention provides a nucleic acid and a lipid nanoparticle including the intranasal formulation and the lipid nanoparticle composition.

In some embodiments, the nucleic acid is encapsulated by the lipid nanoparticle composition. The nucleic acid may include an antisense oligonucleotide, a siRNA, a miRNA, a self-amplifying RNA (SAM samRNA or saRNA), a self-replicating DNA, an LNA, a DNA, a replicon, an mRNA, a guide RNA, a transposon, a single gene, a vector, a plasmid, a complex of RNA and RNA-binding protein, or a combination thereof. In some embodiments, the nucleic acid includes an antigenic encoded mRNA.

In some embodiments, the lipid nanoparticle has a diameter of about 50 nm to about 700 nm.

In some embodiments, the lipid nanoparticle has a polydispersity index of about 0.01 to about 0.80.

In some embodiments, the lipid nanoparticle has an encapsulation efficiency of about 50% to about 100%.

In another aspect, the invention provides a pharmaceutical composition including the lipid nanoparticle and a pharmaceutically acceptable carrier.

In yet another aspect, the invention provides a method for preparing the lipid nanoparticle or the pharmaceutical composition. The method includes:
(i) forming a lipid nanoparticle by mixing the nucleic acid and the lipid nanoparticle composition including the ionizable lipid, structural lipid, sterol, and stabilizing agent in a microfluidic mixer;
(ii) purifying the lipid nanoparticle; and
(iii) combining the purified lipid nanoparticle with the intranasal formulation.

In yet another aspect, the invention provides a use of the lipid nanoparticle or the pharmaceutical composition for preventing, treating, or ameliorating conditions or diseases including administering the lipid nanoparticle or the pharmaceutical composition to lung tissues.

The embodiments of the invention were used to make unique solutions with adhesive or sticky properties almost like saliva or mucous to hold the nanoparticles in the nasal cavity or lungs epithelium and to resist fast draining.

The combined components of the solutions of the invention acts to non-destructively adhere nanoparticles to the epithelial wall of the cavity or lungs, act as a penetration enhancer to deliver the nanoparticles into the epithelial barriers to access the lungs cells or immune cells, and foam to protect the LNP or nanoparticle structure from shear stress during aerosolization or nebulization, preserve LNP structure during freeze-thaw, and to prevent precipitation of nucleic acids or antigenic protein.

Further and alternative aspects and features of the disclosed principles will be appreciated from the following detailed description. As will be appreciated, the compositions and methods disclosed herein are capable of being carried out and used in other and different embodiments, and capable of being modified in various respects. Accordingly, it is to be understood that both the foregoing general description and the following detailed description are exemplary and explanatory only and do not restrict the scope of the appended claims.

### BRIEF DESCRIPTION OF THE DRAWINGS

In drawings which illustrate embodiments of the invention,

FIG. 1A is a bar graph showing size (nm) and PDI of the LNPs after mixing with intranasal (IN) formulations F32, F33, F34, F35 at a volume ratio of 1:1, with measurements taken at day 1, 4 °C before being subjected to freezing and storage at −80 °C, and at day 8 of storage at −80 °C.

FIG. 1B Is a bar graph showing encapsulation efficiency (%) and loading concentration (μg/mL) of the 1:1 mixture of IN formulations F32, F33, F34, F35 and LNPs, with measurements taken at day 8 of storage at −80 °C.

FIG. 1C is a bar graph showing size (nm) and PDI of the LNPs after mixing with intranasal (IN) formulations F36 to F51 at a volume ratio of 1:1, with measurements taken at day 1, 4 °C before being subjected to freezing and storage at −80°, and at day 8 of storage at −80 °C.

FIG. 1D Is a bar graph showing encapsulation efficiency (%) and loading concentration (μg/mL) of the 1:1 mixture of IN formulations F36 to F51 and LNPs, with measurements taken at day 8 of storage at −80 °C.

FIG. 2A is a bar graph illustrating size and PDI for mixture of IN formulations F32, F33, F34 and F35 and LNPs.

FIG. 2B Is a bar graph showing encapsulation efficiency (%) and loading concentration (μg/mL) of the lyophilized LNPs, where the LNPs were mixed with IN formulations F32, F33, F34, F35 at 1:1 volume ratio prior to lyophilization.

FIG. 3A is a line graph showing relative % of GFP expressing BHK cells after being treated with lyophilized LNPs in IN formulations F32, F33, F34, F35, compared to non-lyophilized and freshly prepared LNP control in the absence of IN formulations.

FIG. 3B shows comparison of fluorescent images of the eGFP expression in the BHK cells at 24 h after treatment with non-lyophilized and freshly prepared LNP alone (left) or with the LNPs lyophilized with IN formulations (right). The lyophilized LNP samples were reconstituted with 1×PBS before imaging.

FIG. 4A is a bioluminescent imaging of four mice under anaesthetic which have received Fluc mRNA-containing LNP with cryobuffer (CB1) but not treated with an IN formulation (Control). No IN formulation was used in the control samples.

FIG. 4B is a bioluminescent imaging of four mice under anaesthetic which have all received test samples (LNPs treated with IN formulation F32), showing pulmonary localization of the test samples.

FIG. 5A is a bar graph showing the total flux (p/s) values of two controls, PBS and empty LNP, and four test samples (LNPs with IN formulations F32, F33, F34, F35, respectively), after administration into mice and visualization using the bioluminescence reader.

FIG. 5B is a bar graph showing the individual lung weights (grams) for two controls (PBS, empty LNP) and treated mice from the same experiment of FIG. 5A.

FIG. 6A is a schematic representation that shows the SARS-CoV-2 vaccination study in mice. In this study, mice were injected with SARS-CoV-2 expressing LNPs at day 1 and day 28. Plasma serum was collected at day 21 and day 42 and SARS-CoV-2 antigen specific IgG levels were measured. Pre-bleed was performed before vaccination.

FIG. 6B is a dot plot showing the relative humoral immune responses (IgG) in the plasma serum of vaccinated female Balb/c mice at day 21 and day 42. Mice received the IN vaccinations using COVID-19-specific saRNA (A14)-encapsulated IN-specific LNPs (A14-LNP1, A14-LNP2, and A14-LNP3, respectively). A volume of 12.5 μL dose of each IN vaccine was administered into each nostril of the mouse by pipette drop. LNP# represents different PNI proprietary ionizable lipids and may be selected from a list of non-limiting examples of ionizable lipids, according to descriptions of the disclosure.

FIG. 6C is a dot plot showing the relative humoral immune responses (IgG) in the plasma serum of vaccinated female Balb/c mice at day 21 and day 42. Mice received the IN vaccinations using COVID-19-specific saRNA (A14 and A5)-encapsulated IN-specific LNPs (A14-LNP3 and A5-LNP3, respectively). A volume of 12.5 μL dose of each IN vaccine was administered into each nostril of the mouse by pipette drop. LNP# represents different PNI proprietary ionizable lipids and may be selected from a list of non-limiting examples of ionizable lipids, according to descriptions of the disclosure.

FIG. 6D is a dot plot showing the relative humoral immune responses (IgG) in the plasma serum of vaccinated female Balb/c mice at day 21 and day 42. Mice received the COVID-19-specific saRNA (A5)-encapsulated IN-specific LNPs via intranasal (IN) or intramuscular (IM) routes (A5-LNP3 (IN) and A5-LNP3 (IM), respectively). A volume of 12.5 μL dose of each IN vaccine was administered into each nostril of the mouse by pipette drop. LNP# represents different PNI proprietary ionizable lipids and may be selected from a list of non-limiting examples of ionizable lipids, according to descriptions of the disclosure.

FIG. 6E is a dot plot showing the relative humoral mucosal immune responses (IgA) in the plasma serum of vaccinated female Balb/c mice at day 21 and day 42. Mice received the IN or IM vaccinations using COVID-19-specific saRNA (A14 and A5)-encapsulated IN-specific LNPs (A14-LNP1 (IN), A14-LNP2 (IN), A14-LNP3 (IN), A5-LNP1 (IN), and A5-LNP3 (IM), respectively). A volume of 12.5 μL dose of each IN vaccine was administered into each nostril of the mouse by pipette drop. LNP# represents different PNI proprietary ionizable lipids and may be selected from a list of non-limiting examples of ionizable lipids, according to descriptions of the disclosure.

FIG. 6F is a dot plot showing the relative body weight changes (%) of all the vaccinated mice groups until day 42. Mice received the IN or IM vaccinations using saRNA-encapsulated IN-specific LNPs (A14-LNP1 (IN), A14-LNP2 (IN), A14-LNP3 (IN), A5-LNP1 (IN), and A5-LNP3 (IM), respectively). A volume of 12.5 μL dose of each IN vaccine was administered into each nostril of the mouse by pipette drop. LNP# represents different PNI proprietary ionizable lipids and may be selected from a list of non-limiting examples of ionizable lipids, according to descriptions of the disclosure.

FIG. 7 is a bar graph showing the size and PDI of the liposomal formulations after mixing with intranasal (IN) formulation F32 at a volume ratio of 1:1, with measurements taken at day 0, 4 °C before being subjected to freezing, at day 8 of storage at 4 °C, and at day 8 of storage at −80 °C.

### DETAILED DESCRIPTION

To facilitate an understanding of the present disclosure, a number of terms and phrases are defined below.

Unless otherwise defined, all technical and scientific terms used herein have the same meaning as commonly understood by one of ordinary skill in the art to which the methods described herein belong. Any reference to standard methods refers to the most recent available version of the method at the time of filing of this disclosure unless otherwise indicated.

For any method disclosed herein that includes discrete steps, the steps may be conducted in any feasible order. And, as appropriate, any combination of two or more steps may be conducted simultaneously.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the invention.

The terms “comprising,” “having,” “including,” and “containing” are to be construed as open-ended terms (i.e., meaning “including, but not limited to,”) unless otherwise noted. Such terms will be understood to imply the inclusion of a stated step or element or group of steps or elements but not the exclusion of any other step or element or group of steps or elements.

The singular form “a”, “an” and “the” include plural referents unless the context clearly dictates otherwise. These articles refer to one or to more than one (i.e., to at least one). The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. As used herein, the term “or” is generally employed in its usual sense including “and/or” unless the content clearly dictates otherwise. The term “and/or” means any one or more of the items in the list joined by “and/or”. As an example, “x and/or y” means any element of the three-element set {(x), (y), (x, y)}. In other words, “x and/or y” means “one or both of x and y”. As another example, “x, y, and/or z” means any element of the seven-element set {(x), (y), (z), (x, y), (x, z), (y, z), (x, y, z)}. In other words, “x, y and/or z” means “one or more of x, y and z”.

Where ranges are given, endpoints include all numbers subsumed within that range (e.g., 1 to 5 includes 1, 1.5, 2, 2.75, 3, 3.80, 4, 5, etc.). Furthermore, unless otherwise indicated or otherwise evident from the context and understanding of one of ordinary skill in the art, values that are expressed as ranges can assume any specific value or subrange within the stated ranges in different embodiments of the disclosure, to the tenth of the unit of the lower limit of the range, unless the context clearly dictates otherwise. Herein, “up to” a number (for example, up to 50) includes the number (for example, 50). The term “in the range” or “within a range” (and similar statements) includes the endpoints of the stated range.

Reference throughout this specification to “one aspect (or embodiment),” “an aspect (or embodiment),” “certain aspects (or embodiments),” or “some aspects (or embodiments),” etc., means that a particular feature, configuration, composition, or characteristic described in connection with the aspect is included in at least one aspect of the disclosure. Thus, the appearances of such phrases in various places throughout this specification are not necessarily referring to the same embodiment of the disclosure. Furthermore, the particular features, configurations, compositions, or characteristics may be combined in any suitable manner in one or more aspects.

Unless otherwise indicated, all numbers expressing quantities of components, molecular weights, and so forth used in the specification and claims are to be understood as being modified in all instances by the term “about.” As used herein in connection with a measured quantity, the term “about” refers to that variation in the measured quantity as would be expected by the skilled artisan making the measurement and exercising a level of care commensurate with the objective of the measurement and the precision of the measuring equipment used. The term “about” as used in connection with a numerical value throughout the specification and the claims denotes an interval of accuracy, familiar and acceptable to a person skilled in the art. In general, such interval of accuracy is +/−10%. Thus, “about” can be understood to be within 10%, 9%, 8%, 7%, 6%, 5%, 4%, 3%, 2%, 1%, 0.5%, 0.1%, 0.05%, 0.01%, or 0.001% greater or less than the stated value. Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

The term “exemplary” means serving as a non-limiting example, instance, or illustration. As utilized herein, the terms “e.g.,” and “for example” set off lists of one or more non-limiting aspects, examples, instances, or illustrations.

As used herein, the term “substantially” refers to the qualitative condition of exhibiting total or near-total extent or degree of a characteristic or property of interest. Biological and chemical phenomena rarely, if ever, go to completion and/or proceed to completeness or achieve or avoid an absolute result. The term “substantially” is therefore used herein to capture the potential lack of completeness inherent in many biological and chemical phenomena. For example, “substantially” may refer to being within at least about 20%, alternatively at least about 10%, alternatively at least about 5% of a characteristic or property of interest.

The term “administering” as used herein refers to the physical introduction of an agent to a subject, such as a lipid nanoparticle disclosed herein, using any of the various methods and delivery systems known to those skilled in the art. Exemplary routes of administration for the formulations disclosed herein include intravenous, intramuscular, subcutaneous, intraperitoneal, spinal, or other parenteral routes of administration, for example by injection or infusion. The phrase “parenteral administration” means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. In some embodiments, the formulation is administered via a non-parenteral route, e.g., orally. Other non-parenteral routes include a topical, epidermal or mucosal route of administration, for example, intranasally, vaginally, rectally, sublingually or topically. Administering can also be performed, for example, once, a plurality of times, and/or over one or more extended periods.

The term “cancer” refers to a broad group of various diseases characterized by the uncontrolled growth of abnormal cells in the body. Unregulated cell division and growth results in the formation of malignant tumors that invade neighboring tissues and may also metastasize to distant parts of the body through the lymphatic system or bloodstream. A “cancer” or “cancer tissue” can include a tumor.

The term “in vitro” refers to events occurring in an artificial environment, e.g., in a test tube, reaction vessel, cell culture, etc., rather than within a multi-cellular organism. The term “in vitro cell” refers to any cell which is cultured ex vivo. In particular, an in vitro cell can include a T cell. The term “in vivo” refers to events that occur within a multi-cellular organism, such as a human or a non-human animal.

The term “nucleic acid” refers to any polymeric chain of nucleotides. A nucleic acid may be DNA, RNA, or a combination thereof. In some embodiments, a nucleic acid comprises one or more natural nucleic acid residues. In some embodiments, a nucleic acid comprises of one or more nucleic acid analogs. In some embodiments, nucleic acids are prepared by one or more of isolation from a natural source, enzymatic synthesis by polymerization based on a complementary template (in vivo or in vitro), reproduction in a recombinant cell or system, and chemical synthesis. In some embodiments, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long (e.g., 20 to 100, 20 to 500, 20 to 1000, 20 to 2000, or 20 to 5000 or more residues). In some embodiments, a nucleic acid is partly or wholly single stranded; in some embodiments, a nucleic acid is partly or wholly double stranded. In some embodiments a nucleic acid has a nucleotide sequence comprising at least one element that encodes, or is the complement of a sequence that encodes, a polypeptide.

The term “pharmaceutically acceptable” refers to a molecule or composition that, when administered to a recipient, is not deleterious to the recipient thereof, or that any deleterious effect is outweighed by a benefit to the recipient thereof. With respect to a carrier, diluent, or excipient used to formulate a composition as disclosed herein, a pharmaceutically acceptable carrier, diluent, or excipient must be compatible with the other ingredients of the composition and not deleterious to the recipient thereof, or any deleterious effect must be outweighed by a benefit to the recipient. The term “pharmaceutically acceptable carrier” means a pharmaceutically-acceptable material, composition or vehicle, such as a liquid or solid filler, diluent, excipient, or solvent encapsulating material, involved in carrying or transporting an agent from one portion of the body to another (e.g., from one organ to another). Each carrier present in a pharmaceutical composition must be “acceptable” in the sense of being compatible with the other ingredients of the formulation and not deleterious to the patient, or any deleterious effect must be outweighed by a benefit to the recipient. Some examples of materials which may serve as pharmaceutically acceptable carriers comprise: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

“Treatment” or “treating” of a subject refers to any type of intervention or process performed on, or the administration of an active agent to, the subject with the objective of reversing, alleviating, ameliorating, inhibiting, slowing down or preventing the onset, progression, development, severity or recurrence of a symptom, complication or condition, or biochemical indicia associated with a disease. In one embodiment, “treatment” or “treating” includes a partial remission. In another embodiment, “treatment” or “treating” includes a complete remission. In some embodiments, treatment may be of a subject who does not exhibit signs of the relevant disease, disorder and/or condition and/or of a subject who exhibits only early signs of the disease, disorder, and/or condition. In some embodiments, such treatment may be of a subject who exhibits one or more established signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of the relevant disease, disorder, and/or condition.

A “disease”, as used herein, is a state of health of a subject wherein the subject cannot maintain homeostasis, and wherein if the disease is not ameliorated, the subject's health continues to deteriorate. In contrast, a “disorder” is a state of health in which the subject is able to maintain homeostasis, but in which the subject's state of health is less favorable than it would be in the absence of the disorder. Left untreated, a disorder does not necessarily cause a further decrease in the subject's state of health. A disease or disorder is “alleviated” if the severity of a sign or symptom of the disease or disorder, the frequency with which such a sign or symptom is experienced by a subject, or both, is reduced.

As used herein, the terms “subject”, “individual”, and “patient” are interchangeable, and relate to vertebrates, preferably mammals. For example, mammals in the context of the disclosure are humans, non-human primates, domesticated animals such as dogs, cats, sheep, cattle, goats, pigs, horses, etc., laboratory animals such as mice, rats, rabbits, guinea pigs, etc., as well as animals in captivity such as animals in zoos. The term “animal” as used herein includes humans. The term “subject” may also include a patient, i.e., an animal, having a disease. In exemplary aspects, a subject, individual, or patient refers to a human (e.g., a man, a woman, or a child).

As used herein, the term “preventing a disease” in a subject means, for example, to stop the development of one or more clinical symptoms of a disease or disorder in a subject before they occur or are detectable. Preferably, the disease or disorder does not develop at all, i.e., no symptoms of the disease or disorder are detectable. In some aspects, it can also mean delaying or slowing of the development of one or more symptoms of the disease or disorder. Alternatively, or in addition, it can mean decreasing the severity of one or more subsequently developed symptoms.

The advantages of the invention are that the IN formulations enable intranasal (IN) administration, aerosol inhalation, and intratracheal instillation of nucleic acid-encapsulated nanoparticles including, but not limited to, LNPs, liposomes, and polymeric nanoparticles, to directly access the pulmonary system, principally lung epithelium, and avoid off-target effects in other organs.

The intranasal formulation of the invention includes a viscosity agent, an epithelial adherence agent, and a foaming agent.

In some embodiments, the IN formulations are used with a lipid nanoparticle composition or a nucleic acid-containing lipid nanoparticles (NALNP). The NALAP comprises lipid nanoparticles including a nucleic acid encapsulated therein. Lipid nanoparticles are a subgroup of lipid particles with a mean diameter of from about 15 to about 500 nm, e.g., about 15 nm, about 25 nm, about 50 nm, about 75 nm, about 100 nm, about 125 nm, about 150 nm, about 175 nm, about 200 nm, about 225 nm, about 250 nm, about 275 nm, about 300 nm, about 325 nm, about 350 nm, about 375 nm, about 400 nm, about 425 nm, about 450 nm, about 475 nm, or about 500 nm, or a diameter defined by a range of any two of the foregoing values. In some embodiments, the mean particle diameter is greater than 200 nm. In some embodiments, the lipid particle has a diameter of about 300 nm or less, 250 nm or less, 200 nm or less, 150 nm or less, 100 nm or less, or 50 nm or less. In one embodiment, the lipid particle has a diameter of from about 50 to about 150 nm. Smaller particles generally exhibit increased circulatory lifetime in vivo compared to larger particles. Smaller particles have an increased ability to reach tumor sites than larger nanoparticles. In one embodiment, the lipid particle has a diameter from about 15 to about 150 nm.

Lipid nanoparticles are generally spherical assemblies of lipids, sterols, and stabilizing agents. Positive and negative charges, ratios, as well as hydrophilicity and hydrophobicity dictate the physical structure of the lipid particles in terms of size and orientation of components. The structural organization of these lipids may lead to an aqueous interior with a minimum bilayer as in liposomes or it may have a solid interior as in a lipid nanoparticle. There may be phospholipid monolayers or bilayers in single or multiple forms. Lipid particles are between 1 and 1000 nm in size. In some embodiments, lipid nanoparticles comprise a lipid nanoparticle composition (or referred to interchangeably as “lipid composition” throughout the disclosure) and a nucleic acid. In some embodiments, lipid nanoparticle composition comprises an ionizable lipid, a structural lipid, a sterol, and a stabilizing agent. “N/P” is the ratio of moles of the amine groups of ionizable lipids to those of the phosphate groups of nucleic acid. Any suitable N/P ratio can be used to combine the lipid nanoparticle composition and the nucleic acid. In some embodiments, the lipid nanoparticle composition and the nucleic acid are combined at a N/P ratio from about 2 to about 20, e.g., about 2, about 3, about 4, about 5, about 6, about 7, about 8, about 9, about 10, about 11, about 12, about 13, about 14, about 15, about 16, about 17, about 18, about 19, or about 20, or at an N/P ratio defined by a range of any two of the aforementioned values. In some case, the N/P ratio is more than about 1, more than about 2, more than about 3, more than about 4, more than about 5, more than about 6, more than about 7, more than about 8, or more than about 9. In some case, the N/P ratio is less than about 40, less than about 38, less than about 36, less than about 34, less than about 32, less than about 30, less than about 28, less than about 26, less than about 24, less than about 22, less than about 20, less than about 18, less than about 16, less than about 14, less than about 12, or less than about 10. In a preferred embodiment, the N/P ratio is 6-10. For example, in preferred embodiments, the N/P ratio is 6, 8, or 10. The nucleic acid is associated with the lipid composition to form a nucleic acid-containing LNP in a premeditated ratio such as ionizable lipid amine (N) to nucleic acid phosphate ration (P) of N/P 4, N/P 6, N/P 8, N/P 10, N/P 12 or any other suitable N/P ratio.

In some embodiments, a lipid composition comprises an ionizable lipid. Any suitable ionizable lipid can be used in embodiments of the present invention. An ionizable lipid is a lipid that is cationic or becomes ionizable (protonated) as the pH is lowered below the pKa of the ionizable group of the lipid but is more neutral at higher pH values. At pH values below the pKa, the lipid is then able to associate with negatively charged nucleic acids (e.g., oligonucleotides). Examples of suitable ionizable lipids are found in PCT Publication Nos. WO2020252589 and WO2021000041.

In some embodiments, the ionizable lipid is DODMA (1,2-dioleyloxy-3-dimethylaminopropane), DLin-MC3-DMA (O—(Z,Z,Z,Z-heptatriaconta-6,9,26,29-tetraen-19-yl)-4-(N,N-dimethylamino)), DLin-KC2-DMA (2-dilinoleyl-4-dimethylaminoethyl-[1,3]-dioxolane), butanoic acid, BOCHD-C3-DMA (4-(dimethylamino)-,9-(2-octylcyclopropyl)-1-[8-(2 octylcyclopropyl) octyl]nonyl ester), or C12-200. In some preferred embodiments, the ionizable lipid is (Z)-3-(2-((1,17-bis(2-octylcyclopropyl)heptadecan-9-yl)oxy)-2-oxoethyl)-2-(pent-2-en-1-yl)cyclopentyl 4-(dimethylamino)butanoate (referred to as PNI 516) (WO 2020/252589) or (2R,3S,4R)-2-(((1,4-dimethylpiperidine-4-carbonyl)oxy)methyl)tetrahydrofuran-3,4-diyl (9E,9′E,12E,12′E)-bis(octadeca-9,12-dienoate) (referred to as PNI 127) (WO 2021/000041).

In some embodiments a lipid composition comprises a structural lipid in addition to a phophoester or phosphoramidite. Representative structural lipids include diacylphosphatidylcholines, diacylphosphatidylethanolamines, diacylphosphatidylglycerols, ceramides, sphingomyelins, dihydrosphingomyelins, cephalins, and cerebrosides. Exemplary structural lipids include zwitterionic lipids, for example, distearoylphosphatidylcholine (DSPC), dioleoylphosphatidylcholine (DOPC), dipalmitoylphosphatidylcholine (DPPC), dioleoyl-phosphatidylethanolamine (DOPE), palmitoyloleoylphosphatidylcholine (POPC), 1-stearoyl-2-oleoyl-sn-glycero-3-phosphocholine (SOPC), palmitoyloleoyl-phosphatidylethanolamine (POPE) and dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidylethanolamine (DSPE), 16-O-monomethyl PE, 16-O-dimethyl PE, 18-1-trans PE, 1-stearoyl-2-oleoyl-phosphatidyethanol amine (SOPE), and 1,2-dielaidoyl-sn-glycero-3-phophoethanolamine (trans DOPE). In one preferred embodiment, the structural lipid is distearoylphosphatidylcholine (DSPC).

In another embodiment, the structural lipid is any lipid that is negatively charged at physiological pH. These lipids include phosphatidylglycerols such as dioleoylphosphatidylglycerol (DOPG), dipalmitoylphosphatidylglycerol (DPPG), palmitoyloleyolphosphatidylglycerol (POPG), cardiolipin, phosphatidylinositol, diacylphosphatidylserine, diacylphosphatidic acid, and other anionic modifying groups joined to neutral lipids. Other suitable structural lipids include glycolipids (e.g., monosialoganglioside GM1).

In some embodiments, the lipid composition comprises a sterol. Any suitable sterol can be used. In some embodiments, the sterol is cholesterol, beta-sitosterol, 20-alpha-hydroxysterol, phytosterol, or a combination thereof. In a preferred embodiment, the sterol is cholesterol.

“Stabilizer” or stabilizing agent is a term used to identify the agent that is added to the ionizable lipid, the structural lipid, and the sterol that form the lipid formulation according to embodiments of the invention. Non-limiting examples of non-ionic stabilizing agents include: Polyethyleneglycol (PEG), DMG-PEG2000 (1,2-dimyristoyl-rac-glycero-3-methoxypolyethylene glycol-2000) Polysorbates (Tweens), TPGS (Vitamin E polyethylene glycol succinate), Brij™ S20 (polyoxyethylene (20) stearyl ether), Brij™35 (Polyoxyethylene lauryl ether, Polyethyleneglycol lauryl ether), Brij™S10 (Polyethylene glycol octadecyl ether, Polyoxyethylene (10) stearyl ether), polyglycerol, and Myrj™52 (polyoxyethylene (40) stearate).

The inventive lipid composition of the instant disclosure may comprise any suitable combination of ionizable lipid, structural lipid, sterol, and the stabilizing agent. In some embodiments, the lipid composition comprises 47.5 mol % ionizable lipid, 12.5 mol % structural lipid, 38.5 mol % sterol, and 1.5 mol % stabilizing agent. In preferred embodiments, the lipid composition comprises 20 to 70 mol % ionizable lipid, and 10 to 50 mol % structural lipid, 20 to 50 mol % cholesterol and 0.1 to 5 mol % of stabilizing agent. In some embodiments, the stabilizing agent includes polyglycerol.

The nucleic acid-containing lipid nanoparticle comprises a nucleic acid. Any suitable nucleic acid can be used in the nucleic acid-containing lipid nanoparticle. The nucleic acid is a substance intended to have a direct or indirect effect in the diagnosis, cure, mitigation, treatment or prevention of disease, or to have direct or indirect effect in restoring, correcting or modifying physiological functions, or to act as a research reagent. In some embodiments, the nucleic acid is a siRNA, miRNA, a self-amplifying RNA (SAM or saRNA), a self-replicating DNA, an LNA, a DNA, replicon, an mRNA, a guide RNA, a single guide RNA, circular RNA, a transposon, a single gene, or a combination thereof. In some embodiments, the nucleic acid is referred to as a nucleic acid therapeutic or NAT. In some embodiments, the nucleic acid represents an antigen encoded mRNA for prophylactic or therapeutic vaccine.

In some embodiments, the NALNP is in an anhydrous form. In some embodiments, the NALNP is in an anhydrous form consists of a lyophilized or lyo cake. In some embodiments, the NALNP is in a reconstituted form. In a reconstituted form, a lyophilized NALNP has had a pharmaceutically acceptable diluent added to the lyophilized NALNP as described herein.

In some embodiments, the LNP may include a liposomal composition for delivery of small molecules. In embodiments, encapsulation of DiR; DiIC18(7) (1,1′-Dioctadecyl-3,3,3′,3′-Tetramethylindotricarbocyanine Iodide) dye mimicking small molecule or similar therapeutic compounds in liposomal formulations and their 1:1 mixture with lead IN compositions are used. The near IR fluorescent, lipophilic carbocyanine DiOC18(7) (‘DiR’) is weakly fluorescent in water but highly fluorescent and quite photostable when incorporated into membranes.

Any suitable method of mixing can be used to form the lipid nanoparticle. The lipid nanoparticles according to embodiments of the invention can be prepared by standard T-tube mixing techniques, turbulent mixing, titration mixing, agitation promoting orders self-assembly, or passive mixing of all the elements with self-assembly of elements into nanoparticles. A variety of methods have been developed to formulate lipid nanoparticles containing genetic drugs. Suitable methods are disclosed in U.S. Pat. No. 5,753,613 by Ansell, Mui and Hope and U.S. Pat. No. 6,734,171 by Saravolac et al., by way of example. These methods include mixing preformed lipid particles with nucleic acids in the presence of ethanol or mixing lipid dissolved in ethanol with an aqueous media containing nucleic acid and result in lipid nanoparticles with nucleic acid encapsulation efficiencies of 65-99%. Both of these methods rely on the presence of ionizable lipid to achieve encapsulation of nucleic acid and a stabilizing agent to inhibit aggregation and the formation of large structures.

Automatic micro-mixing instruments such as the NanoAssemblr® instruments (Precision NanoSystems Inc, Vancouver, Canada) enable the rapid and controlled manufacture of nanomedicines (liposomes, lipid nanoparticles, and polymeric nanoparticles). NanoAssemblr® instruments accomplish controlled molecular self-assembly of nanoparticles via microfluidic mixing cartridges that allow millisecond mixing of nanoparticle components at the nanoliter, microlitre, or larger scale with customization or parallelization. Rapid mixing on a small scale allows reproducible control over particle synthesis and quality that is not possible in larger instruments.

Preferred methods incorporate instruments such as the microfluidic mixing devices like the NanoAssemblr® Spark™, Ignite™, Benchtop™ and NanoAssemblr® Blaze™ in order to achieve nearly 100% of the nucleic acid used in the formation process is encapsulated in the particles in one step. In one embodiment, the lipid particles are prepared by a process by which from about 90 to about 100% of the nucleic acid used in the formation process is encapsulated in the particles.

U.S. Pat. Nos. 9,758,795 and 9,943,846, by Cullis et al. describe methods of using small volume mixing technology and novel formulations derived thereby. U.S. Pat. No. 10,159,652 by Ramsay et al. describes more advanced methods of using small volume mixing technology and products to formulate different materials. U.S. Pat. No. 9,943,846 by Walsh, et al. discloses microfluidic mixers with different paths and wells to elements to be mixed PCT Publication WO2017117647 by Wild, Leaver and Taylor discloses microfluidic mixers with disposable sterile paths. U.S. Pat. No. 10,076,730 by Wild, Leaver and Taylor discloses bifurcating toroidal micromixing geometries and their application to micromixing. PCT Publication No. WO2018006166 by Chang, Klaassen, Leaver et al. discloses a programmable automated micromixer and mixing chips therefore. U.S. Design Nos. D771834, D771833, D772427, and D803416 by Wild and Leaver, and U.S. Design Nos. D800335, D800336 and D812242 by Chang et al., disclose mixing cartridges having microchannels and mixing geometries for mixer instruments sold by Precision NanoSystems ULC.

In embodiments of the invention, devices for biological microfluidic mixing are used to prepare the lipid particles according to embodiments of the invention. The devices include a first and second stream of reagents, which feed into the microfluidic mixer, and lipid particles are collected from the outlet, or emerge into a sterile environment.

The first stream includes a therapeutic agent in a first solvent. Suitable first solvents include solvents in which the therapeutic agents are soluble and that are miscible with the second solvent. Suitable first solvents include aqueous buffers. Representative first solvents include citrate and acetate buffers or other low pH buffers.

The second stream includes lipid composition in a second solvent. Suitable second solvents include solvents in which the ionizable lipids according to embodiments of the invention are soluble, and that are miscible with the first solvent. Suitable second solvents include 1,4-dioxane, tetrahydrofuran, acetone, acetonitrile, dimethyl sulfoxide, dimethylformamide, acids, and alcohols. Representative second solvents include aqueous ethanol 90%, or anhydrous ethanol.

In one embodiment of the invention, a suitable device includes one or more microchannels (i.e., a channel having its greatest dimension less than 1 mm). In one example, the microchannel has a diameter from about 20 to about 300 μm. In examples, at least one region of the microchannel has a principal flow direction and one or more surfaces having at least one groove or protrusion defined therein, the groove or protrusion having an orientation that forms an angle with the principal direction (e.g., a staggered herringbone mixer), as described in U.S. Pat. No. 9,943,846, or a bifurcating toroidal flow as described in U.S. Pat. No. 10,076,730. To achieve maximal mixing rates, it is advantageous to avoid undue fluidic resistance prior to the mixing region. Thus, one example of a device has non-microfluidic channels having dimensions greater than 1000 μm, to deliver the fluids to a single mixing channel.

Less automated mixing methods and instruments such as those disclosed in Zhang, S-h et al., Chemical Eng. J. 144(2): 324-328 (2008), and Stroock A et al., U.S. Published Patent Application US20040262223, and Jeffs, L B et al., Pharm. Resch., 22(3): 362-372 (2005)), are also useful in creating lipid particle compositions of the invention.

Any suitable flow ratio can be used to combine the lipid nanoparticle composition and the nucleic acid. In some embodiments, the lipid nanoparticle composition and the nucleic acid are combined using a flow ratio of about 1:1 (or 1) to about 10:1 (or 10) (aqueous phase:organic phase) by volume, e.g., about 1, about 2, about 3, about 4, about 5, about 6, or about 7, about 8, about 9, about 10, or a flow ratio defined by a range of any two of the aforementioned values. In some cases, the flow ratio is more than about 0.5:1. In some cases, the flow ratio is less than about 20, less than about 18, less than about 16, less than about 14, less than about 12, less than about 10, or less than about 8. Any suitable total flow rate can be used to combine the lipid nanoparticle composition and the nucleic acid. In some embodiments, the lipid nanoparticle composition and the nucleic acid are combined with a total flow rate of the organic phase and aqueous phase from about 2 to about 2000 mL/min, e.g., about 2 mL/min, about 4 mL/min, about 6 mL/min, about 8 mL/min, about 10 mL/min, about 20 mL/min, about 40 mL/min, about 60 mL/min, about 80 mL/min, or about 100 mL/min, about 120 mL/min, about 140 mL/min, about 160 mL/min, about 180 mL/min, about 200 mL/min, about 220 mL/min, about 240 mL/min, about 260 mL/min, about 280 mL/min, about 300 mL/min, about 350 mL/min, about 400 mL/min, about 450 mL/min, or about 500 mL/min, about 550 mL/min, about 600 mL/min, about 650 mL/min, about 700 mL/min, about 750 mL/min, about 800 mL/min, about 850 mL/min, or about 900 mL/min, about 950 mL/min, about 1000 mL/min, about 1100 mL/min, about 1200 mL/min, about 1300 mL/min, about 1400 mL/min, about 1500 mL/min, about 1600 mL/min, about 1700 mL/min, about 1800 mL/min, about 1900 mL/min, about 2000 mL/min, or a total flow rate defined by a range of any two of the foregoing values. In some cases, the total flow rate is more than about 1 mL/min, 2 mL/min, 4 mL/min, 6 mL/min, 8 mL/min, 10 mL/min, 20 mL/min, or 40 mL/min. In some case, the total flow rate is less than about 3000 mL/min, less than about 2800 mL/min, less than about 2600 mL/min, less than about 2400 mL/min, less than about 2200 mL/min, less than about 2100 mL/min, less than about 2000 mL/min, less than about 1800 mL/min, less than about 1600 mL/min, less than about 1500 mL/min, less than about 1400 mL/min, less than about 1200 mL/min, less than about 1000 mL/min, or less than about 800 mL/min. In some embodiments, the lipid nanoparticle composition and the nucleic acid are combined using a flow ratio from about 1:1 (or 1) to about 10:1 (or 10) by volume (aqueous phase:organic phase) at a N/P ratio from about 2 to about 20, and a total flow rate from about 2 to about 2000 mL/min. In some embodiments, the flow ratio is 3 (aqueous phase:organic phase) to optimize for a particular payload or molar ratio of lipid components.

In some embodiments of the invention, the addition of single or multiple amino acid in IN formulation is used to mix with the lipid nanoparticles at various ratio including 1:1.

In some embodiments, a nucleic acid payload is encapsulated by an exemplary lipid nanoparticle composition. Any suitable nucleic acidpayload can be present in the lipid nanoparticle composition. The payload may include a nucleic acid. In some cases, the payload may include a therapeutic agent. The nucleic acid may be a substance intended to have a direct effect in the diagnosis, cure, mitigation, treatment or prevention of disease, or to have direct effect in restoring, correcting or modifying physiological functions, or to act as a research reagent. Exemplary nucleic acids include any oligonucleotide or polynucleotide whose delivery into a cell causes a desirable effect. The nucleic acid can be single-stranded DNA or RNA, or double-stranded DNA or RNA, or DNA-RNA hybrids, or contain portions of both double stranded and single stranded sequence, as appropriate. In some embodiments, the lipid nanoparticle comprises one or more nucleic acids, two or more nucleic acids, three or more nucleic acids, or four or more nucleic acids. Including more than one nucleic acid may be beneficial in some embodiments (e.g., gene editing). In some embodiments, the nucleic acid is an antisense oligonucleotide, a siRNA, a miRNA, a self-amplifying RNA (samRNASAM or saRNA), a self-replicating DNA, an LNA, a DNA, a replicon, an mRNA, a guide RNA, a transposon, a single gene, a vector, a plasmid, a complex of RNA and RNA-binding protein(s), or a combination thereof. In some embodiments, the nucleic acid is an antigen encoded mRNA.

In some embodiments, the lipid nanoparticle composition is a therapeutic composition, such as an mRNA-based therapeutic composition. The therapeutic composition may optionally include one or more therapeutically acceptable carriers, diluents, or excipients such as salts, buffering agents, preservatives, antiadherents, antioxidants, binders, coatings, compression aids, disintegrants, dyes, emollients, emulsifiers, fillers, film formers or coatings, flavors, fragrances, glidants, lubricants, sorbents, suspending or dispersing agents, sweeteners, waters of hydration, and/or other therapeutic agents. As used herein, the term “excipient” means any pharmaceutically acceptable additive, carrier, diluent, adjuvant, or other ingredient, other than the active pharmaceutical ingredient (API) (and typically in addition to components of the delivery vehicle compositions), suitably selected with respect to the intended form of administration, and consistent with conventional pharmaceutical practices. The disclosed compounds can be administered to a subject or patient in a therapeutically effective amount. The complexes can be administered alone or as part of a pharmaceutically acceptable composition or formulation. In addition, the compositions can be administered all at once, as for example, by a bolus injection, multiple times, or delivered substantially uniformly over a period of time. It is also noted that the dose of the compound can be varied over time.

In some embodiments, the lipid nanoparticle composition may encapsulate an antigen encoded mRNA and be used as is a vaccine. In some embodiments, the antigen encoded mRNA is for a prophylactic or therapeutic vaccine. A vaccine may be referred to as a substance used to stimulate the production of antibodies and provide immunity against one or several diseases, prepared from the causative agent of a disease, its products, or a synthetic substitute. The vaccine may further comprise one or more immunologic adjuvants. As used herein, the term “immunologic adjuvant” refers to a compound or a mixture of compounds that acts to accelerate, prolong, enhance or modify immune responses when used in conjugation with an immunogen (e.g., neoantigens). Adjuvant may be non-immunogenic when administered to a host alone, but that augments the host's immune response to another antigen when administered conjointly with that antigen. Specifically, the terms “adjuvant” and “immunologic adjuvant” are used interchangeably in the present disclosure. Adjuvant-mediated enhancement and/or extension of the duration of the immune response can be assessed by any method known in the art including without limitation one or more of the following: (i) an increase in the number of antibodies produced in response to immunization with the adjuvant/antigen combination versus those produced in response to immunization with the antigen alone; (ii) an increase in the number of T cells recognizing the antigen or the adjuvant; and (iii) an increase in the level of one or more cytokines. Adjuvants may be aluminum based adjuvants including but not limiting to aluminum hydroxide and aluminum phosphate; saponins such as steroid saponins and triterpenoid saponins; bacterial flagellin and some cytokines such as GM-CSF. Adjuvants selection may depend on antigens, vaccines, and routes of administrations.

In some aspects, adjuvants improve the adaptive immune response to a vaccine antigen by modulating innate immunity or facilitating transport and presentation. Adjuvants act directly or indirectly on antigen presenting cells (APCs) including dendritic cells (DCs). Adjuvants may be ligands for toll-like receptors (TLRs) and can directly affect DCs to alter the strength, potency, speed, duration, bias, breadth, and scope of adaptive immunity. In other instances, adjuvants may signal via proinflammatory pathways and promote immune cell infiltration, antigen presentation, and effector cell maturation. This class of adjuvants includes mineral salts, oil emulsions, nanoparticles, and polyelectrolytes and comprises colloids and molecular assemblies exhibiting complex, heterogeneous structures. In one example, the composition further comprises pidotimod as an adjuvant. In another example, the composition further comprises CpG as an adjuvant.

In some embodiments, the lipid nanoparticle and the pharmaceutically acceptable carrier are a pharmaceutical composition. Examples of pharmaceutically acceptable carrier are provided herein.

### EXAMPLES

The following examples further illustrate the invention but should not be construed as in any way limiting its scope.

All solvents and reagents were commercial products and used as such unless noted otherwise.

Temperatures are given in degrees Celsius. The following abbreviations are used with respect to the Examples:

### FLuc: Firefly Luciferase

The FLuc is an mRNA that expresses a luciferase protein, originally isolated from the firefly, in cells. FLuc is commonly used in mammalian cell culture to measure both gene expression and cell viability. It emits bioluminescence in the presence of the substrate, luciferin. FLuc is polyadenylated and optimized for mammalian systems. It mimics a fully processed mature mRNA.

### CMC: Carboxymethyl Cellulose

CMC is a non-toxic cellulose derivative and generally considered as hypoallergenic. It alters rheology, acting as a viscosity agent, of the IN and acts as a binder according to embodiments of the invention. Chemically, CMC is a cellulose derivative and is composed of derivatized glucose joined via β-(1, 4) glycosidic linkages. CMC is formed after cellulose dispersion in alkali followed by treatment with monochloroacetic acid to substitute hydroxyl groups of glucose at positions 0-2, 0-3, or 0-6.

### PLL: Poly-L Lysine

PLL is a food preservative against yeast, fungi, gram-positive, and gram-negative bacteria. It may be used as an epithelial adherence agent for the lipid nanoparticles.

### GH: Gelatin Hydrosylate

GH is the result of gelatin hydrolysis. GH may be extracted from bovines, fish, and pigs by the hydrolysis of gelatin in the animals' tissues. It has been widely used in immunoassay, as an excipient in vaccine formulation to screen its ability to stabilize influenza vaccine (e.g., Flumist). It can act as a foaming agent and is generally regarded as safe.

eGFP-mRNA: Enhanced green fluorescent protein mRNA, which is a basic (constitutively fluorescent) green fluorescent protein derived from Aequorea Victoria.
h: Hour(s)
HPLC: High performance Liquid Chromatography
LNP: lipid nanoparticles
MFI: Median Fluorescence Intensity
min: Minute(s)
mL: Milliliter(s)
mm: Millimeter(s)
mmol: Millimole(s)
NALNP: nucleic acid containing lipid nanoparticles.
PDI: polydispersity index, or a measure of the size range of LNP
μL: Micro Liter(s)
PBS: Phosphate Buffer saline
wt: Weight
C: Degrees Celsius
CB1: sucrose in Tris buffer and 1×PBS (10 mg/mL). CB1 acts as a cryoprotectant agent.
iL: Ionizable Lipid
IVIS: In Vivo Imaging System (veterinary imaging technology)
HL: Helper/Structural Lipid
IN: Intranasal
IM: Intramuscular
saRNA: Self-amplifying mRNA
PEG-DMG: 1,2-dimyristoyl-rac-glycero-3-methoxypolyethylene glycol-2000, 2509 Da, from Avanti)
A5: SARS-Cov-2 spike protein encoded saRNA using V101 vector
A14: SARS-Cov-2 spike protein encoded saRNA
PNI 516 is ionizable lipid (Z)-3-(2-((1,17-bis(2-octylcyclopropyl) heptadecan-9-yl) oxy)-2-oxoethyl)-2-(pent-2-en-1-yl) cyclopentyl 4-(dimethylamino)butanoate;
PNI 580 is ionizable lipid (2S,3R,4R)-2-(((4-(dimethylamino)butanoyl)oxy)methyl)tetrahydrofuran-3,4-diyl bis(2-hexyldecanoate);
PNI 728 is ionizable lipid ((2R,3R,4S)-3,4-bis((2-hexyldecyl)oxy)tetrahydrofuran-2-yl)methyl 4-(diethylamino)butanoate;
PNI 726 is ionizable lipid (2R,3S,4S)-2-((3-(dimethylamino)propoxy)methyl)tetrahydrofuran-3,4-diyl bis(2-hexyldecanoate);
PNI 730 is ionizable lipid (2R,3S,4S)-2-((2-(dimethylamino)ethoxy)methyl)tetrahydrofuran-3,4-diyl bis(2-hexyldecanoate), and
DiR: DiIC18(7) (1,1′-Dioctadecyl-3,3,3′,3′-Tetramethylindotricarbocyanine Iodide).

### Example 1

### Preparation of LNP and NALNP

Components of the lipid compositions include: ionizable lipid “iL” (e.g., PNI 516, PNI 580, PNI 726, PNI 728, PNI 730), structural or helper lipid (e.g., DSPC, DOPE, DOPC, DPPC), sterol (e.g., cholesterol, β-sitosterol), and stabilizing (PEG-DMG or alternate stabilizers) agents in different molar ratios. Lipid compositions (e.g., 25 mM) were prepared in anhydrous ethanol by combining prescribed amounts of lipids from individual lipid stocks in ethanol following defined ratio of components (e.g., exemplary lipid compositions composition as defined in Tablet). The aqueous phase was prepared by diluting nucleic acid (NA) such as mRNA/saRNA/pDNA solutions in 100 mM sodium acetate buffer (pH 4). NALNPs were then prepared by running the lipid composition and NA through a microfluidic mixer (e.g., NanoAssemblr® Ignite™ microfluidic mixer with an Ignite™ NxGen™ (DVBM) cartridge, Precision Nanosystems) at a flow ratio (e.g., aqueous phase:organic phase 1:1 to 10:1) at a N/P ratio range (e.g., from 2 to 20), unless otherwise noted. The resulting LNPs were diluted 25 times in 1×PBS (pH 7.4), and the mixture was subjected to downstream processing. Downstream processing included either ethanol removal through dialysis (e.g., in PBS pH 7.4, or using Amicon™ centrifugal filters (Millipore, USA) at 2500 RPM, or using tangential flow filtration systems). By these methods, the nanoparticles were concentrated to a target dose. Finally, the concentrated LNPs were mixed with IN formulation with a volume ratio (e.g., a volume ratio of 1:1) and kept at −80 °C until use.

TABLE 1
Exemplary lipid compositions

| Composition ID | Ratio in mol % |
|---|---|
| V02 | 47.5 mol % IL/12.5 mol HL/38.5 mol % |
| | Chol/1.5 mol % Stabilizing agent |
| V62 | 40.0 mol % IL/12.5 mol % HL/46 mol % |
| | Chol/1.5 mol % Stabilizing agent |
| V00 | 50 mol % IL/10 mol % HL/38.5 mol % |
| | Chol/1.5 mol % Stabilizing agent |
| V22 | 40 mol % IL/20 mol % HL/37.5 mol % |
| | Chol/2.5 mol % Stabilizing agent |
| V90c | 48 mol % IL/13 mol % HL/39 mol % Chol |
| V46 | 28.7 mol % IL/49.8 mol % HL/20 mol % |
| | Chol/1.5 mol % Stabilizing agent |

### Example 2

### Size, Polydispersity Index (PDI) and Encapsulation Efficiency (EE) of NALNPs

Size and PDI of the LNPs were measured by Dynamic Light Scattering (DLS) using a ZetaSizer™ Nano ZS™ (Malvern Instruments). He/Ne laser of 633 nm wavelength was used as the light source. Data were measured from the scattered intensity data conducted in backscattering detection mode (measurement angle=173°). 0.5 to 2 μL of the sample was placed in a cuvette and diluted with PBS (0.3 mL). Measurements were an average of 10 runs of two cycles each per sample. Z-average size in nm was reported as the particle size and is defined as the harmonic intensity averaged particle diameter. Encapsulation Efficiency (EE) of the LNPs was measured by Quant-iT™ RiboGreen® RNA reagent. These LNP characteristics, as well as the results of the nucleic acid EE for the LNPs are described in the following examples.

### Example 3

Different ratios of three, four, five or six components were tested in association with either nucleic acid containing lipid nanoparticles (NALNP) or “naked” nucleic acid. This mixture, in double distilled water, may be referred to as “IN”.

Table 2 lists the tested additives and ratios of various components in the IM formulations. To illustrate the ratios, consider that a 4% weight per volume would be 4 grams in 100 mL of the IN formulation.

Poly(lysine) has Mol. Wt. 15,000-30,000 Da and was purchased from Sigma-Aldrich (Catalog #P7890-1 G).

TABLE 2
Compositions of IN formulations

| | | | | | Su- | Argi- | Leu- | Histi- | Gly- |
|---|---|---|---|---|---|---|---|---|---|
| IN | CMC | PLL | GH | | crose | nine | cine | dine | cine |
| Formula | (%) | (%) | (%) | Tris | (%) | (%) | (%) | (%) | (%) |
| ID | w/v | w/v | w/v | (mM) | w/v | w/v | w/v | w/v | w/v |
| F1 | 2 | 0.6 | 4 | 40 | 20 | — | — | — | — |
| F2 | 1 | 0.3 | 2 | 40 | 20 | — | — | — | — |
| F3 | 0.5 | 0.15 | 1 | 40 | 20 | — | — | — | — |
| F4 | 1 | 0.3 | 4 | 40 | 20 | — | — | — | — |
| F5 | 0.5 | 0.15 | 4 | 40 | 20 | — | — | — | — |
| F6 | 2 | 0.6 | 4 | — | 10 | — | — | — | — |
| F7 | 1 | 0.3 | 2 | — | 10 | — | — | — | — |
| F8 | 0.5 | 0.15 | 1 | — | 10 | — | — | — | — |
| F9 | 1 | 0.3 | 4 | — | 10 | — | — | — | — |
| F10 | 0.5 | 0.15 | 4 | — | 10 | — | — | — | — |
| F11 | 2 | 0.6 | 4 | — | 20 | — | — | — | — |
| F12 | 1 | 0.3 | 2 | — | 20 | — | — | — | — |
| F13 | 0.5 | 0.15 | 1 | — | 20 | — | — | — | — |
| F14 | 1 | 0.3 | 4 | | 20 | — | — | — | — |
| F15 | 0.5 | 0.15 | 4 | — | 20 | — | — | — | — |
| F16 | 1 | 0.02 | 4 | 40 | 20 | — | — | — | — |
| F17 | 0.5 | 0.02 | 4 | 40 | 20 | — | — | — | — |
| F18 | 1 | 0.01 | 4 | 40 | 20 | — | — | — | — |
| F19 | 0.5 | 0.01 | 4 | 40 | 20 | — | — | — | — |
| F20 | 1 | 0.04 | 4 | 40 | 20 | — | — | — | — |
| F21 | 1 | 0.06 | 4 | 40 | 20 | — | — | — | — |
| F22 | 1 | 0.08 | 4 | 40 | 20 | — | — | — | — |
| F23 | 1 | 0.1 | 4 | 40 | 20 | — | — | — | — |
| F24 | 1 | 0.12 | 4 | 40 | 20 | — | — | — | — |
| F25 | 1 | 0.15 | 4 | 40 | 20 | — | — | — | — |
| F26 | 0.5 | 0.04 | 4 | 40 | 20 | — | — | — | — |
| F27 | 0.5 | 0.06 | 4 | 40 | 20 | — | — | — | — |
| F28 | 0.5 | 0.08 | 4 | 40 | 20 | — | — | — | — |
| F29 | 0.5 | 0.1 | 4 | 40 | 20 | — | — | — | — |
| F30 | 0.5 | 0.12 | 4 | 40 | 20 | — | — | — | — |
| F31 | 0.5 | 0.15 | 4 | 40 | 20 | — | — | — | — |
| F32 | 1 | 0.01 | 4 | 40 | 20 | — | — | — | — |
| F33 | 0.5 | 0.01 | 4 | 40 | 20 | — | — | — | — |
| F34 | 0.25 | 0.01 | 4 | 40 | 20 | — | — | — | — |
| F35 | 0.125 | 0.01 | 4 | 40 | 20 | — | — | — | — |
| F36 | 0.5 | 0.01 | 4 | 40 | 20 | 0.05 | — | — | — |
| F37 | 0.5 | 0.01 | 4 | 40 | 20 | 0.1 | — | — | — |
| F38 | 0.5 | 0.01 | 4 | 40 | 20 | 0.2 | — | — | — |
| F39 | 0.5 | 0.01 | 4 | 40 | 20 | 0.4 | — | — | — |
| F40 | 0.5 | 0.01 | 4 | 40 | 20 | — | 0.05 | — | — |
| F41 | 0.5 | 0.01 | 4 | 40 | 20 | — | 0.1 | — | — |
| F42 | 0.5 | 0.01 | 4 | 40 | 20 | — | 0.2 | — | — |
| F43 | 0.5 | 0.01 | 4 | 40 | 20 | — | 0.4 | — | — |
| F44 | 0.5 | 0.01 | 4 | 40 | 20 | — | — | 0.05 | — |
| F45 | 0.5 | 0.01 | 4 | 40 | 20 | — | — | 0.1 | — |
| F46 | 0.5 | 0.01 | 4 | 40 | 20 | — | — | 0.2 | — |
| F47 | 0.5 | 0.01 | 4 | 40 | 20 | — | — | 0.4 | — |
| F48 | 0.5 | 0.01 | 4 | 40 | 20 | — | — | — | 0.05 |
| F49 | 0.5 | 0.01 | 4 | 40 | 20 | — | — | — | 0.1 |
| F50 | 0.5 | 0.01 | 4 | 40 | 20 | — | — | — | 0.2 |
| F51 | 0.5 | 0.01 | 4 | 40 | 20 | — | — | — | 0.4 |

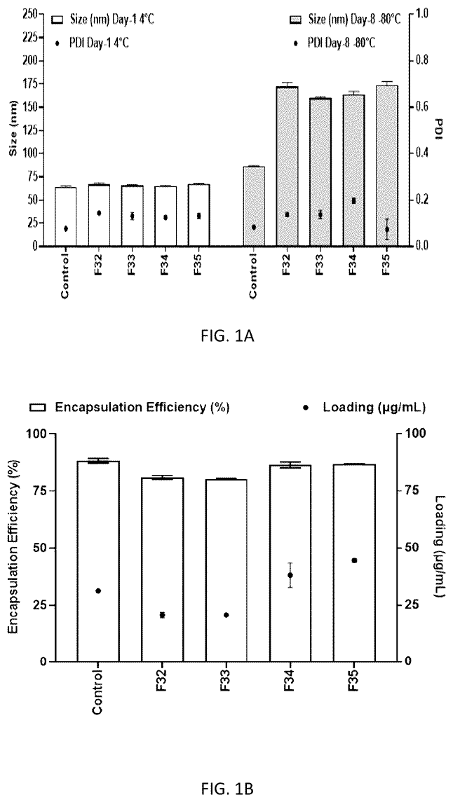

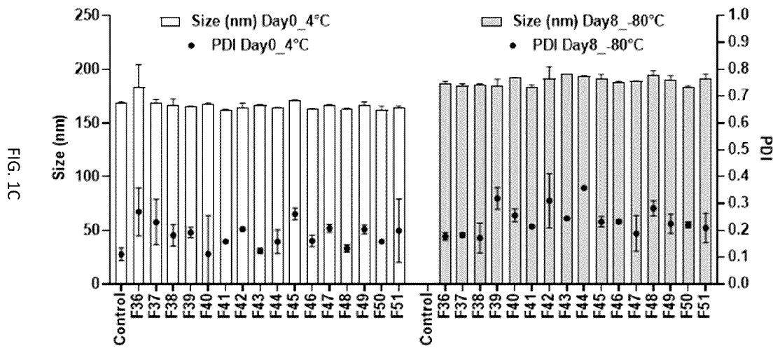

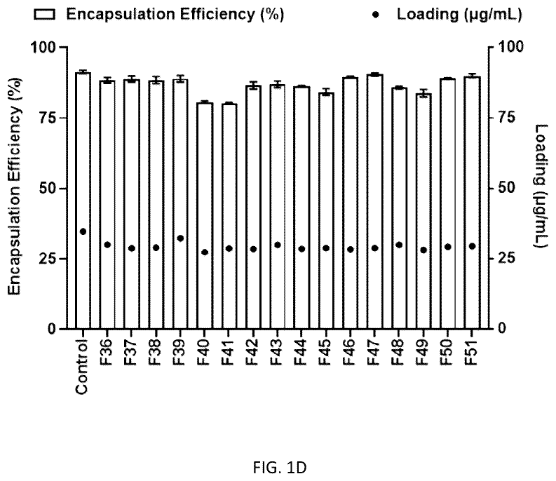

The size and PDI of the LNPs after mixing with IN formulation at a volume ratio of 1:1 at day-1 before freezing and at 8 days of storage at −80 degrees C are graphed in FIG. 1A. There is good sizing, PDI (vertical bars or blocks within graphed columns) and consistency for all tested conditions on Day 1 at 4 degrees C. On the right side of the graph, size and PDT at Day 8 at −80 °C showed size increase for all conditions including control, and PDI fluctuation for F35. Higher CMC content tended to yield higher PDI, however, there were not significant differences between the groups.

EE and loading concentration of the 1:1 mixture of IN formulation and LNPs at 8 days of storage at −80 °C are shown in FIG. 1B. Day 1 is not shown. EE and loading remained steady at over 75% for all of the conditions.

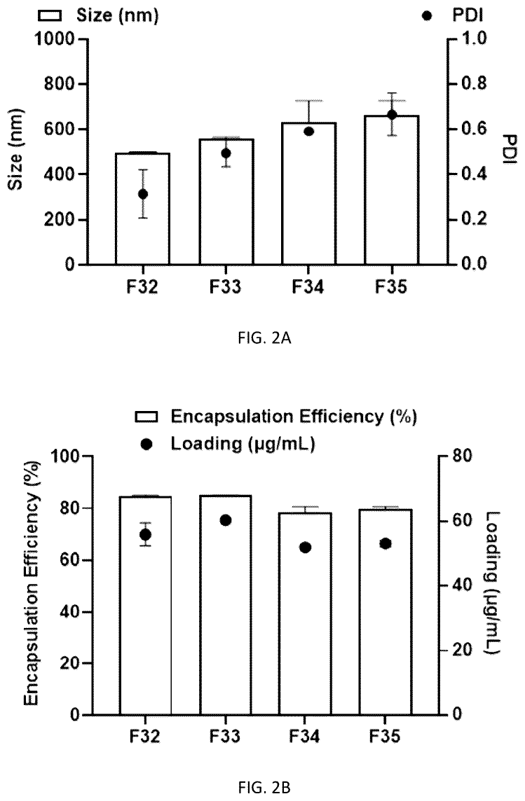

In another experiment, LNPs were mixed with IN formulation at 1:1 ratio prior to lyophilization. Lyophilized LNPs were reconstituted with 1×PBS with IN F32, F33, F34, and F35, respectively. The results for this experiment are shown for size and PDI (FIG. 2A) and EE and loading concentration (FIG. 2B). The size increase after lyophilization is considered acceptable for intranasal and pulmonary application.

### Example 4

eGFP Expression in BHK Cells

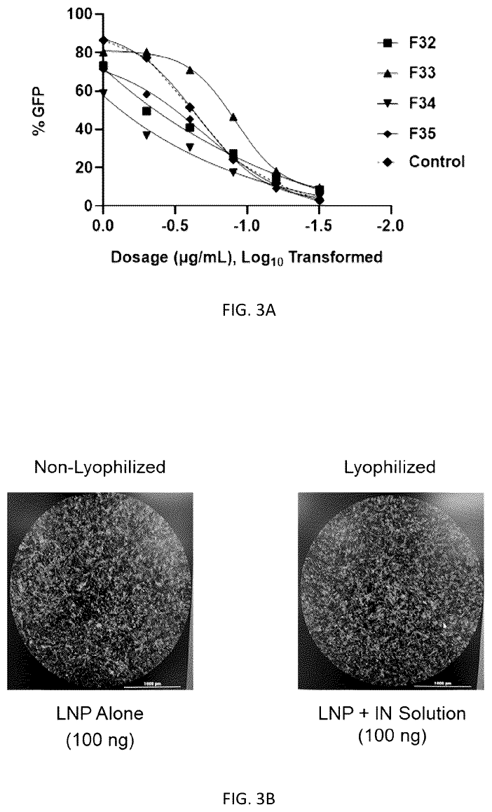

After lyophilization with IN formulations, eGFP-encapsulated LNPs were tested in BHK (baby hamster kidney cells (ATCC via Cedarlane) for transfection efficiencies. The cells were seeded onto Agilent black 96-well plates with clear bottoms at 2,500-5,000 cells per well density. Cells were transfected with the test samples 48 h post seeding. The concentration of eGFP-encapsulated LNPs ranges were varied from 1 μg/ml to 0.3125 ug/ml. The plates were read 24 h post transfection using a Cytation-7 microplate reader. The relative percentages (%) of GFP expressing BHK cells after being treated with lyophilized LNPs in IN formulations F32, F33, F34, F35 were compared to non-lyophilized and freshly prepared LNP control in the absence of IN formulations are shown in FIG. 3A. Logarithmic scaling was used to show the non-linear fit based on the transformed data. The dose response curve (sigmoidal) is illustrated by converting ug/mL to Log concentration.

The relative % of GFP expressing BHK cells after treating with lyophilized LNPs at various concentrations compared to non-lyophilized and freshly prepared LNP control are shown in the representative images of the treated BHK cells at 24 h timepoint of FIG. 3B. For these images, lyophilized LNPs were reconstituted with 1×PBS before imaging. Green fluorescence comparison for the eGFP expression in BHK healthy cells treated with LNP alone (not lyophilized) and LNP+IN (lyophilized) does not show toxicity or any cell viability reduction. Similar level of cell viability and similar level of eGFP expression are observed for LNP alone and LNP+IN formulations.

### Example 5

### Efficacy of IN Formulation Administration in Animals

Balb/C Mice were obtained from Envigo (Indianapolis, IN) and were anesthetized by inhalation of isoflurane until no longer responsive to a toe pinch. The anesthetized animals were removed from the anesthesia chamber and held using their front teeth in an upright position on an intubation stand with the body further supported with gauze taped to the intubation stand and placed across the abdomen. The tongue was positioned so that it was not blocking the air way or injured with light pressure applied from a cotton swab. The test sample was administered in a drop-wise manner using pipette, in 2 doses of 12.5 μL, to each of the animal's nostrils. The total dose each animal received in each nostril was 25 μL. Once the animal had inhaled the first dose of 12.5 μL in each nostril completely, the second dose of 12.5 μL was applied to each nostril. Total dose of 25 μL that the mice received includes 0.1 mg/kg Fluc-mRNA, more specifically, 2 ug payload in total of 25 uL volume. The animals were then released from the intubation stand following complete inhalation of the test sample and maintained, on anesthesia, in a head-up supine position on an approximately 45-degree angle for five more minutes. Animals were then observed until recovery from the anesthetic and monitored periodically for mortality and morbidity. Animals were weighed every weekday and prior to any other procedures for the length of the study. If signs of severe toxicity were observed, the animals were entitled to be euthanized, and a necropsy would be performed to assess other signs of toxicity. The organs including liver, gall bladder, spleen, lung, kidney, heart, intestine, lymph nodes and bladder were examined. Any other unusual findings were also recorded.

Four hours after the administration of test samples, mice were weighed, and administered intraperitoneally with the D-luciferin substrate solution at a dose of 150 mg/kg (200 L/20 g mouse). Mice in the same dosing groups (n=4) were anesthetized with isoflurane and were placed on the imaging platform in a supine position while being maintained on isoflurane via a nose cone. Bioluminescence imaging studies were conducted. Mice were imaged at 12 min post administration of D-luciferin using an exposure time of 1 s, 5 s, 10 s, 30 s, and 60 s. If a single mouse in any group had significantly lower bioluminescence than the other mice in the group, it was imaged alone in addition to the group imaging using exposure times of 30 s and 1 min. Similarly, if a single mouse had a bioluminescence that saturated the camera, it was imaged separately to the other mice in the group using exposure times of 1 s and 5 s. The remaining mice were also reimaged using the original exposure times (10 s and 30 s), so that non-saturated images of all mice were obtained. Immediately following the last imaging timepoint of each group of mice, the mice in that group were euthanized, and blood, liver, lung, spleen, kidney, heart (or areas with any other hot spots as indicated in the bioluminescence images) weighed, collected and (for solid organs) rinsed with PBS prior to being imaged ex vivo using exposure times of 1 s, 5 s, 10 s, and 30 s.

For in vivo imaging, the bioluminescence values were quantified by measuring photon flux (photons/second) and average radiance (p/s/cm2/sr) in the whole animal, and for any focal regions of interest (likely liver and lung regions) using the Living Image® software program.

The liver, spleen, and lung were imaged separately from other organ types. For blood testing, 500 μL of subject blood was placed into a labelled tissue culture plate for imaging. Tissues were weighed after being imaged ex vivo. For ex vivo imaging, the bioluminescence values were quantified by measuring photon flux (photons/second) and by flux/g or flux/mL for each excised organ/sample. The IVIS imaging time points were 6 h, 24 h, 48 h, 72 h, and 96 h.

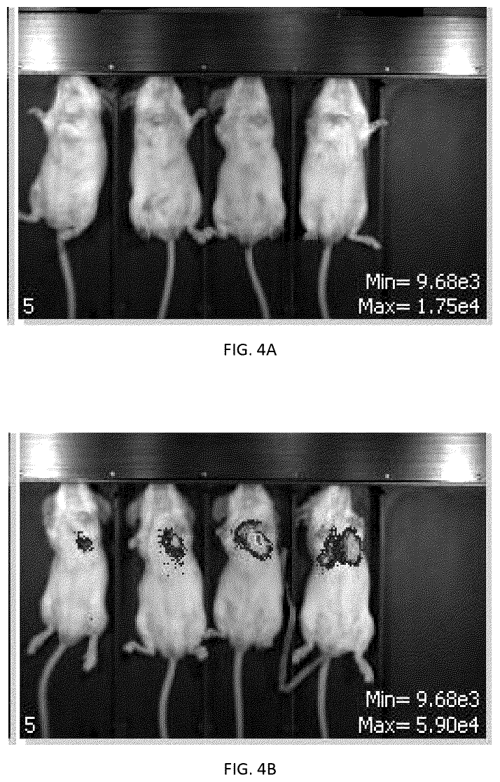

Representative whole body IVIS images were taken of the female Balb/c mice of two groups where one is the control group treated with Fluc-mRNA/LNPs (FIG. 4A) and the other is one of the test groups (F32) receiving 1:1 Fluc-mRNA/LNPs: IN formulation (FIG. 4B).

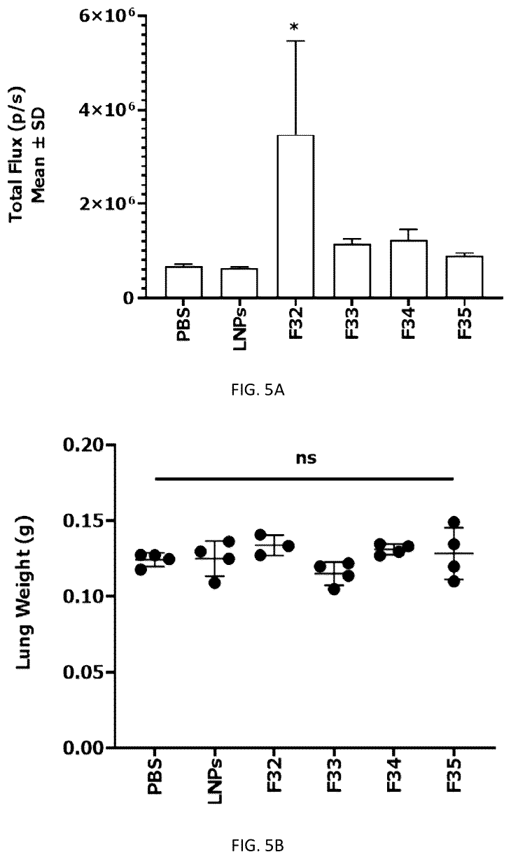

The analysis for the comparative efficacy of the treatment groups after a bioluminescence imaging (e.g., IVIS imaging here) is shown in FIG. 5A. Relative lungs weights for the mice groups are shown in FIG. 5B. In FIG. 5A, y-axis is total flux measured in total number of photons emitted second (p/s) from a region of interest (ROI) during the IVIS imaging, which quantifies the intensity of light emitted from luciferase-expressing cells. Total flux is a direct indicator of the activity of the bioluminescent reporter, which is often linked to biological events, such as gene expression (e.g., luciferase) or cellular activity. Among the IN formulation tested (4F32-F35), F32 showed significantly higher Luc+ expression level than other groups. In FIG. 5B, there was no significant difference for the lung weights in treated mice.

In summary, the prepared and concentrated Fluc-mRNA-loaded LNPs were in the desired size range of 60-80 nm with a PDI value below 0.2 and with an encapsulation efficiency greater than 95% prior to mixing with the IN mucoadhesive solution (INMS). When mixed with INMS at a 1:1 ratio, the average size and PDI of the LNPs were increased to ~175 nm and ~0.3, respectively. In addition, the encapsulation efficiencies of the mixed formulations declined by not less than 80%, as illustrated in, for example, FIG. 1B. After lyophilization, the average size and PDI of the LNPs were increased to ~700 nm and ~0.7, respectively, yet still within acceptable range for intended applications for lyophilized LNPs, and encapsulation efficiencies were unchanged. Our preclinical investigations exhibited significant in vivo luciferase expression in the lungs of female BALB/c mice receiving intranasally administered Fluc-mRNA LNPs mixed with INMS at a 1:1 ratio. There were no clinical symptoms or adverse effects observed. The lead INMS candidate is currently being used for preclinical investigations with aspect to mucosal immunization potentials.

### Example 6

### Vaccine Study in Mice

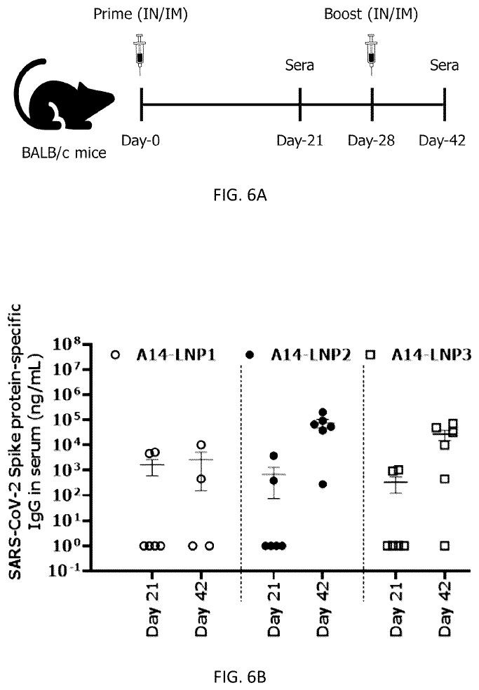

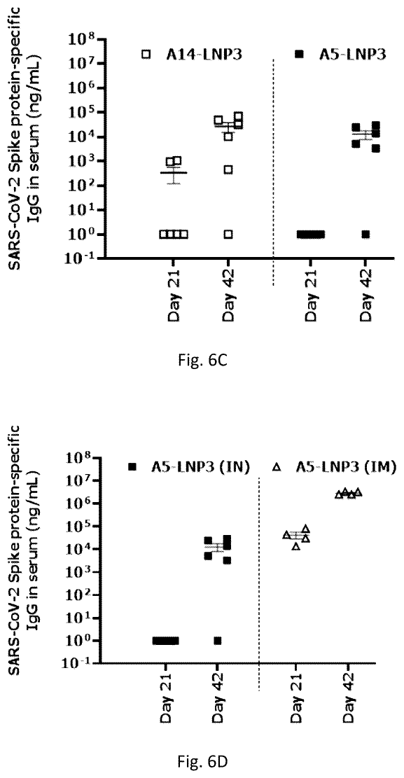

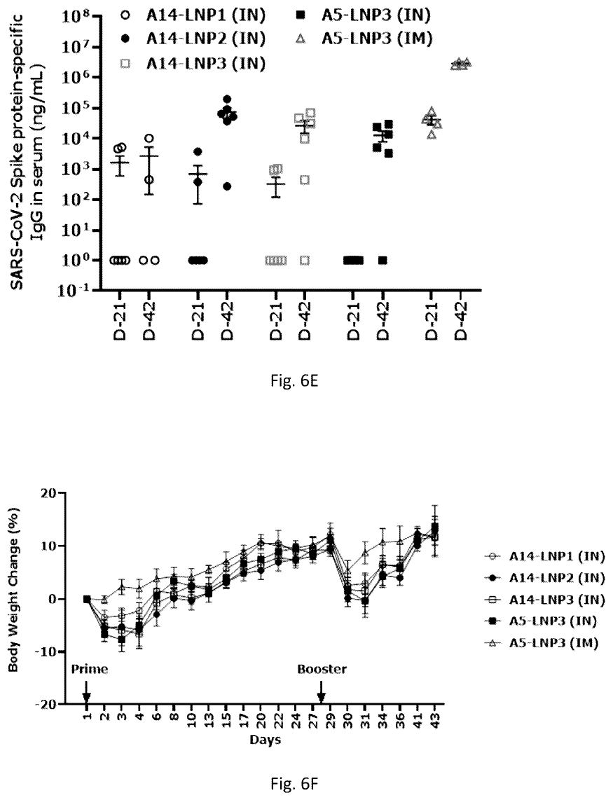

This example describes the procedure used for the SARS-CoV-2 expression evaluation of SARS-CoV-2 expressing A14 and A5 saRNA-LNPs, in vivo. LNPs are either intranasally (IN groups) with IN formulation or intramuscularly (IM group) without IN formulation injected into groups of four mice (N=4, 6-week-old female BALB/c mice) at a prime dose of 0.05 mg/kg (1 μg/50 μL/20-g mouse) on Day 1 and a booster dose of 0.05 mg/kg (1 μg/50 μL/20-g mouse) on Day 28. The sera samples are collected on Day-21 and Day-42. For serum preparation, after collection of the whole blood, the blood was allowed to clot by leaving the collection tube at room temperature for 15-30 minutes. The clot is removed by centrifuging the tubes at 1000-2000×g for 10 min at 4 °C. The clear golden-yellow color supernatant is carefully removed and transferred to sterile screw-capped clear polypropylene tube on ice. The serum is then stored in −80 °C until further use. The SARS-CoV-2 antigen specific IgG and IgA level in sera are determined using enzyme-linked immunoassay (ELISA) and Meso Scale (MSD) assay, respectively. All saRNA-LNPs, when mixed with IN formulation, induced the potent expression of SARS-CoV-2 spike protein specific IgG and IgA titers in mice (FIGS. 6B-6E) along with booster response.

Balb/C Mice were obtained from Envigo (Indianapolis, IN) and were anesthetized by inhalation of isoflurane until no longer responsive to a toe pinch. The anesthetized animals were removed from the anesthesia chamber and held using their front teeth in an upright position on an intubation stand with the body further supported with gauze taped to the intubation stand and placed across the abdomen. The tongue was positioned so that it was not blocking the air way or injured with light pressure applied from a cotton swab. The test sample was administered in a drop-wise manner using pipette, in 2 doses of 12.5 μL, to each of the animal's nostrils. Once the animal had inhaled the first does of 12.5 uL in each nostril completely, the second dose of 12.5 μL was applied to each nostril. Each dose of 12.5 μL that the mice received includes 0.1 mg/kg Fluc-mRNA. The animals were then released from the intubation stand following complete inhalation of the test sample and maintained, on anesthesia, in a head-up supine position on an approximately 45-degree angle for five more minutes. Animals were then observed until recovery from the anesthetic and monitored periodically for mortality and morbidity. Animals were weighed every weekday and prior to any other procedures for the length of the study. If signs of severe toxicity were observed, the animals were entitled to be euthanized, and a necropsy would be performed to assess other signs of toxicity. The organs including liver, gall bladder, spleen, lung, kidney, heart, intestine, lymph nodes and bladder were examined. Any other unusual findings were also recorded.

The vaccination study plan and the analysis for the comparative humoral immune efficacy of the vaccinated groups at day 21 and day 42 is shown in FIGS. 6A-6E. Relative body weight changes for the vaccinated mice groups are shown in FIG. 6F. IN formulation #F33 was used for this vaccination study. There was no significant difference for the body weight changes in vaccinated mice.

In summary, the prepared and concentrated saRNA-encapsulated LNPs were in the desired size range of 60-80 nm with a PDI value below 0.2 and with an encapsulation efficiency greater than 95% prior to mixing with the INMS. When mixed with INMS at a 1:1 ratio, the average size and PDI of the LNPs were increased to ~175 nm and ~0.3, respectively. In addition, the encapsulation efficiencies of the mixed formulations declined by not less than 80%. Our mucosal vaccination study exhibited significant in vivo spike protein expression in the lungs of female BALB/c mice receiving intranasally administered saRNA LNPs mixed with INMS at a 1:1 ratio. There were no clinical symptoms or adverse effects observed. The lead INMS candidate is currently being used for preclinical investigations with aspect to other genetic diseases of lungs and delivery of antigenic proteins as well as small molecules.

### Example 7

### Preparation of Liposomal Formulations for the Delivery of Small Molecules

Components of the lipid composition include structural or helper lipid (e.g., POPC, DSPC, DOTAP), sterol (e.g., cholesterol), and stabilizing agents (e.g., DMG-PEG or alternate stabilizers) in different molar ratios. The lipid composition (e.g., 25 mM) was prepared in anhydrous ethanol by combining prescribed amounts of lipid components from respective individual lipid component stocks in ethanol following ratio as defined for various compositions (e.g., see Table 3). The aqueous phase used was 1×PBS (pH 7.4). Liposomal formulations were then prepared by running the lipid composition and PBS through a microfluidic mixer (e.g., NanoAssemblr® Ignite™ microfluidic mixer with an Ignite™ NxGen™ (DVBM) cartridge, Precision Nanosystems) at a flow ratio (e.g., range from 1:1 to 10:1 aqueous phase: organic phase) at N/P ratio (e.g., range from 2 to 20), unless otherwise noted. The resulting liposomes were diluted 10 times in 1×PBS (pH 7.4), and the mixture was subjected to downstream processing. Downstream processing may include ethanol removal through dialysis in PBS (pH 7.4), using Amicon™ centrifugal filters (Millipore, USA) at 2500-3000 RPM, or using tangential flow filtration systems, or a combination thereof. By these methods, the liposomes were concentrated to a target dose. Finally, the concentrated liposomes were mixed with IN formulation with a predefined volume ratio (e.g., 1:0.5, 1:1, or 1:1.5) and kept at −80 °C until use.

TABLE 3
Exemplary lipid compositions

| Composition | |
|---|---|
| ID | Ratio in mol % |
| L01 | 55 mol % HL/45 mol % Chol |
| L02 | 55 mol % HL/44.75 mol % Chol/0.25 mol % DiR |
| L03 | 55 mol % HL/42 mol % Chol/3 mol % SAF |
| L04 | 55 mol % HL/41.75 mol % Chol/3 mol % |
| | SAF/0.25 mol % DiR |

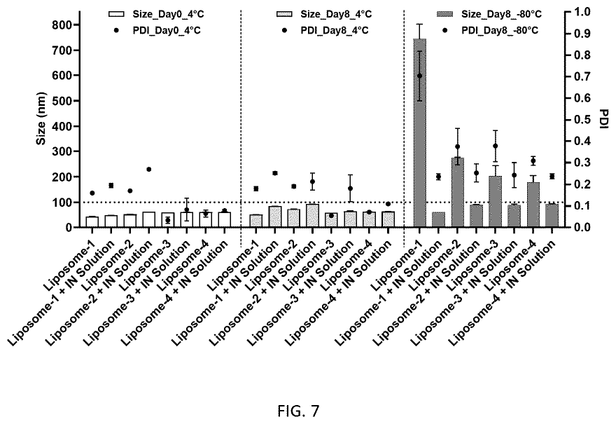

Size and PDI of the liposomal nanoparticles were measured by Dynamic Light Scattering (DLS) using a ZetaSizer™ Nano ZS™ (Malvern Instruments). He/Ne laser of 633 nm wavelength was used as the light source. Data were measured from the scattered intensity data conducted in backscattering detection mode (measurement angle=173°). 0.5 to 2 μL of the sample was placed in a cuvette and diluted with PBS (0.3 mL). Measurements were an average of 10 runs of two cycles each per sample. Z-average size in nm was reported as the particle size and is defined as the harmonic intensity averaged particle diameter. The size and PDI of the liposomes after mixing with IN formulation at a volume ratio of 1:1 at day-0 before freezing and at 8 days of storage at −80 °C were graphed in FIG. 7. There is good sizing, PDI (vertical bars or blocks within graphed columns) and consistency for all tested conditions on Day 0 at 4 °C. On the right side of the graph, size and PDT at Day 8 at −80 °C showed size slightly increased for all conditions including control, and PDI fluctuation for composition including IN formulation. However, composition including IN formulation significantly prevented the size and PDI increase of liposomal formulations after 8 days of storage at −80 °C (see FIG. 7).

All references, including publications, patent applications, and patents, cited herein are hereby incorporated by reference to the same extent as if each reference were individually and specifically indicated to be incorporated by reference and were set forth in its entirety herein.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. An intranasal formulation for intranasal or intrapulmonary administration including a viscosity agent, an epithelial adherence agent, and a foaming agent.

2. The formulation of claim 1, wherein the viscosity agent includes carboxymethylcellulose and/or wherein the epithelial adherence agent includes poly-lysine.

3. (canceled)

4. (canceled)

5. The formulation of claim 1, wherein the foaming agent includes gelatin hydrolysate.

6. The formulation of claim 1, further including a cryoprotectant.

7. The formulation of claim 6, wherein the cryoprotectant includes an emulsifying agent, a sugar, or a combination thereof.

8. The formulation of claim 6, wherein the cryoprotectant includes tris(hydroxymethyl)aminomethane and sucrose.

9. The formulation of claim 1, further including an amino acid, wherein the amino acid is arginine, leucine, histidine, glycine, or a combination thereof.

10. (canceled)

11. The formulation of am claim 1, wherein the formulation includes, based on grams per 100 millimeters (mL) of the formulation (% wt/v), about 0.1 to about 2% wt/v of the viscosity agent, about 0.01 to about 0.6% wt/v of the epithelial adherence agent, and about 1 to about 4 grams % wt/v of the foaming agent.

12. The formulation of claim 11, wherein the formulation further includes sucrose that is about 10 to about 20 grams per 100 mL of the formulation and/or wherein the formulation further includes an amino acid that is about 0.05 to about 0.4 grams per 100 mL of the formulation.

13. (canceled)

14. The formulation of claim 1, wherein the formulation includes an aqueous buffer.

15. The formulation of claim 1, wherein the formulation is administered intranasally or intratracheally.

16. (canceled)

17. The formulation of claim 1 in aerosolized or nebulized form.

18. The formulation of n m claim 1, further including a lipid nanoparticle, liposome, polymeric nanoparticle, or lipomiceller composition.

19. The formulation of claim 18, wherein the lipid nanoparticle, liposome, polymeric nanoparticle, or lipomiceller composition includes a nucleic acid, a peptide, or a protein.

20. A composition for intranasal or intrapulmonary administration including the intranasal formulation of claim 1 and a lipid nanoparticle composition comprising: (a) an ionizable lipid; (b) a structural lipid; (c) a cholesterol; and (d) a stabilizing agent.

21. The composition of claim 20, wherein the ionizable lipid is DODMA, DLin-MC3-DMA, DLin-KC2-DMA, butanoic acid, BOCHD-C3-DMA, C12-200, PNI 127, PNI 516, PNI 580, PNI 726, PNI 728, PNI 730, or a combination thereof.

22. (canceled)

23. (canceled)

24. The composition of claim 20, wherein the structural lipid comprises distearoylphosphatidylcholine, dioleoylphosphatidylcholine, dipalmitoylphosphatidylcholine, dioleoyl-phosphatidylethanolamine, palmitoyloleoylphosphatidylcholine, 1-stearoyl-2-oleoyl-sn-glycero-3-phosphocholine, palmitoyloleoyl-phosphatidylethanolamine, dioleoyl-phosphatidylethanolamine 4-(N-maleimidomethyl)-cyclohexane-1-carboxylate, dipalmitoyl phosphatidyl ethanolamine, dimyristoylphosphoethanolamine, distearoyl-phosphatidylethanolamine, 1,2-dipalmitoyl-sn-glycero-3-phosphoethanolamine-N-methyl, 1,2-dipalmitoyl-sn-glycero-3-phosphoethanolamine-N,N-dimethyl, 1,2-dielaidoyl-sn-glycero-3-phosphoethanolamine, 1-stearoyl-2-oleoyl-phosphatidyethanol amine, 1,2-dielaidoyl-sn-glycero-3-phophoethanolamine, distearoylphosphatidylcholine, dioleoylphosphatidylglycerol, dipalmitoylphosphatidylglycerol, palmitoyloleyolphosphatidylglycerol, cardiolipin, phosphatidylinositol, diacylphosphatidylserine, diacylphosphatidic acid, monosialoganglioside GM1, or a combination thereof.

25. The composition of claim 20, wherein the sterol comprises cholesterol, beta-sitosterol, 20-alpha-hydroxysterol, phytosterol, or a combination thereof.

26. The composition of claim 20, wherein the lipid nanoparticle composition comprises about 25 to about 70 mol % ionizable lipid, about 10 to about 50 mol % structural lipid, about 20 to about 50 mol % sterol, and about 0 to about 3 mol % stabilizing agent.

27. A lipid nanoparticle comprising the composition of claim 20 and a nucleic acid and wherein the nucleic acid is encapsulated by the lipid nanoparticle composition.

28. (canceled)

29. The lipid nanoparticle of claim 27, wherein the nucleic acid comprises an antisense oligonucleotide, a siRNA, a miRNA, a self-amplifying RNA (SAM samRNA or saRNA), a self-replicating DNA, an LNA, a DNA, a replicon, an mRNA, a guide RNA, a transposon, a single gene, a vector, a plasmid, a complex of RNA and RNA-binding protein, or a combination thereof.

30. The lipid nanoparticle of claim 27, wherein the nucleic acid includes an antigenic encoded mRNA.

31. The lipid nanoparticle of claim 27, wherein the lipid nanoparticle has a diameter of about 50 nm to about 700 nm, a polydispersity index of about 0.01 to about 0.80, and/or an encapsulation efficiency of about 50% to about 100%.

32. (canceled)

33. (canceled)

34. A pharmaceutical composition comprising the lipid nanoparticle of claim 27 and a pharmaceutically acceptable carrier.

35. A method for preparing the lipid nanoparticle of claim 27, the method comprising:
(i) forming a lipid nanoparticle mixture by mixing the nucleic acid and the lipid nanoparticle composition including the ionizable lipid, structural lipid, sterol, and stabilizing agent in a microfluidic mixer;
(ii) purifying the lipid nanoparticle mixture to obtain a purified lipid nanoparticle; and
(iii) combining the purified lipid nanoparticle with the intranasal formulation.

36. A method of use of the lipid nanoparticle of claim 27, for preventing, treating, or ameliorating conditions or diseases including administering the lipid nanoparticle or a pharmaceutical composition including the lipid nanoparticle and a pharmaceutically acceptable carrier to lung tissues.
